(19) United States
(12) Patent Application Publication
(10) Pub. No.: US 2025/0331944 A1
(43) Pub. Date: Oct. 30, 2025

(54) COMPONENT INSERTION SYSTEM

(71) Applicants: Jonathan Yerasimides, Louisville, KY (US); Austin Smith, Gilbert, AZ (US)

(72) Inventors: Jonathan Yerasimides, Louisville, KY (US); Austin Smith, Gilbert, AZ (US)

(21) Appl. No.: 18/650,357

(22) Filed: Apr. 30, 2024

(51) Int. Cl. A61B 46/17

(52) U.S. Cl. CPC A61B 46/17

(57) ABSTRACT

This specification describes a system for facilitating the insertion and implantation of a component into an anatomical space. The system includes a pouch for covering the component manufactured of a low-friction material that eases insertion of the component. The pouch may be shaped to facilitate ease of insertion and perforated to facilitate removal thereof.

### BACKGROUND OF THE INVENTION

The system described herein relates generally to a system for aiding insertion of a component into an enclosed space and more specifically to a surgical system for easily inserting an implant or other implement into an anatomical space during a surgical procedure. The various aspects and embodiments of the system disclosed herein provide a novel component insertion system that provides ease of use and simple operation while solving several common problems in the surgical arts.

During most surgical procedures a surgeon may be required to insert various tools and implements into a surgical space, loosely defined as any part of the anatomy that is opened, exposed, or manipulated during a surgery. Orthopedic surgeries, in particular, regularly require surgeons to insert various tools, implements, and implants into the body during the course of the procedures. To cite two exemplary but non-limiting examples, hip replacement surgeries (hip arthroplasty) and shoulder arthroscopy require the use and insertion of a large assortment of tools into an anatomical space, as well as the hip replacement implants and/or devices and shoulder arthroscopic implants and/or devices being implanted into the patient. Both hip arthroplasty and shoulder arthroscopy surgeries significantly reduce pain and improve the joint's function, allowing individuals to resume daily activities with greater ease. As longevity increases, these surgeries are becoming more and more common. Thus any improvement in the surgical process of these important and pain-relieving procedures is highly desirable.

These operations require the skilled and careful insertion and placement of the hip or shoulder implants that necessitate a certain amount of attendant trauma to the surrounding tissue and muscle as the implant is being inserted and positioned. During an anterior hip arthroplasty, for example, an acetabular cup is placed and positioned in the hip socket by manual insertion. This placement has the potential to cause muscle and tissue trauma as well as causing debris build-up when positioning the cup to the precise location prepared in the hip socket. Acetabular caps are often provided with a rough exterior surface to provide grip or purchase when placed in the socket, and this rough surface easily causes damage to surrounding tissue and muscle during insertion. Similarly, some shoulder arthroscopy implants are also bio-integrative. The process of integration of the implant into the body's tissues over time is impeded by the presence of other materials. The implant surface is important for boney ingrowth into the implant, which leads to long-term implant stability and durability.

Additionally, many surgical instruments have edges or textured portions that readily damage surrounding tissue so that great care must be taken when accessing a particular anatomical location to minimize patient trauma. Obviously, these difficulties are manifest in many different types of surgeries, and are not limited to orthopedic procedures.

Furthermore, when inserting an implant or other object into an anatomical space, the operating team must be constantly concerned with contaminants entering the space. While great care is taken to sterilize the various instruments, tools, and components being used in the surgery, contamination and patient infection is still a serious and ongoing issue that adversely impacts patient outcomes and recovery.

Accordingly, there is a need in the art for a system that may be employed during surgeries to minimize patient trauma while facilitating the sterile insertion and use of surgical components.

### SUMMARY OF THE INVENTION

In various aspects and embodiments the invention described herein provides a novel insertion system that utilizes an insertion pouch for surrounding and containing a component or implant that enables a surgeon to minimize trauma to surrounding tissues when inserting the implant. The insertion pouch may in some embodiments be manufactured of a wide variety of low-friction polymer films that are readily sterilized, and that may be readily torn or ripped when the component or implant is suitably positioned and the pouch needs to be removed. Thus the insertion system disclosed provides a sacrificial sterile container for an implant that is removed and discarded once the procedure is completed.

In various embodiments the insertion pouches may be manufactured in a shape or contour that facilitates the insertion of a specific component, implant, or instrument. Pouches may be shaped as open-ended generally rectangular bags, conical bags with a wide open end, or any of a wide variety of shapes and sizes that will accommodate various irregularly shaped components.

In some aspects and embodiments the insertion pouches are provided with perforated portions, or several sections of perforations of varying lengths and frequencies that facilitate the tearing of the pouches into easily removed fragments when desired. The positioning and orientation of perforations along various portions of the pouches may be varied to accommodate a wide variety of components and their respective orientations when placed properly in an anatomical space. Furthermore, the perforations may be curved or angled to accommodate specific component features to allow the pouches to be easily removed during the surgical procedure. Insertion pouches may be manufactured using materials that tear in a defined pattern or patterns, or processed such that tearing occurs in a defined or predictable pattern, with or without perforations.

In various embodiments and aspects the system may be used to provide a sterile pouch that is packaged with a sterilized implant or component inside it during manufacturing, such that the sterile implant and pouch may simply be removed from its packaging material and employed in surgery without the need for unnecessary handling by the surgical team.

In some embodiments the pouches may be comprised of low-friction material that includes a dimpled or textured exterior surface to allow a surgeon or surgical technician to grasp the pouch containing the component being inserted. The textured surface may be provided in a plurality of different textured patterns. In these embodiments the film material employed to produce the pouches must be capable of being manufactured with textured or raised surface features.

As used herein for purposes of the present disclosure, the term “surgical space” can be used to refer to any anatomical space that is accessed or manipulated during a surgery. Furthermore, as used herein for purposes of the present disclosure, the terms “component” or “surgical component” or “implant” can be used to refer to any device or object that is inserted into a surgical space during a surgery. However any of a wide variety of components may be employed with the concepts and embodiments of the system as taught herein without limiting the scope of the invention.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other advantages and features, which characterize the embodiments, are set forth in the claims appended hereto and form a further part thereof. However, for a better understanding of the embodiments and of the advantages and objectives attained through their use, reference should be made to the Drawing Figures and to the accompanying specification, in which there are described exemplary embodiments. This summary is merely provided to introduce a selection of concepts that are further described below in the detailed description, and is not intended to identify key or essential features of the claimed subject matter, nor is it intended to be used in limiting the scope of the claimed subject matter.

### BRIEF DESCRIPTION OF THE DRAWING FIGURES

In the drawings, like reference characters generally refer to the same parts throughout the different views. Also, the drawings are not necessarily to scale, emphasis instead generally being placed upon illustrating the principles of the invention.

FIG. 1 depicts a plan view of an insertion pouch in accordance with various embodiments;

FIG. 1a depicts a plan view of an insertion pouch in accordance with various embodiments;

FIG. 2 depicts a view of an insertion pouch taken along the line 1-1 of FIG. 1 in accordance with various embodiments;

FIG. 3 is a partial detail view of a surgical pouch of the area 3 of FIG. 1 in accordance with various embodiments;

FIG. 4 depicts a view of shoulder anatomy in accordance with various embodiments;

FIG. 5 depicts an isometric view of an suture anchor in accordance with various embodiments;

FIG. 6 depicts a view of an insertion tool in accordance with various embodiments;

FIG. 7 depicts a view of an insertion pouch in accordance with various embodiments;

FIG. 8 depicts a view of an insertion pouch being used in a shoulder in accordance with various embodiments;

FIG. 9 depicts a view of an insertion pouch being used in a shoulder in accordance with various embodiments;

FIG. 10 depicts a view of an insertion pouch being used in a shoulder in accordance with various embodiments;

FIG. 11 depicts a view of an insertion pouch being used in a shoulder in accordance with various embodiments;

FIG. 12 depicts a plan view of an insertion pouch in accordance with various embodiments;

FIG. 13 depicts a plan view of an insertion pouch in accordance with various embodiments;

FIG. 14 depicts a plan view of an insertion pouch in accordance with various embodiments;

FIG. 15 depicts a plan view of an insertion pouch in accordance with various embodiments;

FIG. 16a depicts a plan view of an insertion pouch in accordance with various embodiments;

FIG. 16b depicts a plan view of an insertion pouch in accordance with various embodiments;

FIG. 16c depicts a plan view of an insertion pouch in accordance with various embodiments;

FIG. 16d depicts a plan view of an insertion pouch in accordance with various embodiments;

FIG. 16e depicts a plan view of an insertion pouch in accordance with various embodiments;

FIG. 17 depicts a plan view of an insertion pouch in accordance with various embodiments;

FIG. 18 depicts a plan view of an insertion pouch in accordance with various embodiments;

FIG. 19 depicts a plan view of an insertion pouch in accordance with various embodiments;

FIG. 20 depicts a view of an insertion pouch in accordance with various embodiments;

FIG. 21 depicts a view of an acetabular cup insertion in accordance with various embodiments; and

FIG. 22 depicts a plan view of an acetabular cup insertion in accordance with various embodiments.

### DETAILED DESCRIPTION OF THE PREFERRED EMBODIMENT(S)

Numerous variations and modifications of the apparatuses and embodiments of the disclosed invention will be apparent to one of ordinary skill in the art, as will be readily seen from the detailed description of the system set forth below. Therefore, the invention is not limited to the specific implementations discussed herein.

Furthermore, the embodiments discussed herein will focus on the implementation of the described techniques and apparatuses for a surgical component insertion system. While the embodiments described herein will refer generally to implementation and use of a component insertion system, one of ordinary skill will recognize that the various embodiments disclosed may be practiced with a broad array of equivalent assemblies, devices, materials, and constructions without departing from the scope thereof.

A hip replacement surgery, medically known as total hip arthroplasty, is a procedure performed to replace a damaged or deteriorated hip joint with an artificial implant. This surgery is commonly recommended for individuals experiencing severe hip pain and limited mobility due to conditions such as osteoarthritis, rheumatoid arthritis, avascular necrosis, or fractures that have caused irreversible damage to the hip joint. The procedure is usually conducted under general anesthesia or a combination of regional anesthesia and sedation.

The procedure for a hip arthroplasty may be summarized as follows. The surgeon makes an incision on the anterior, lateral, or posterior hip of a suitable size to access the hip joint. The muscles and tissues thereby exposed are carefully retracted to expose the hip joint. The damaged parts of the hip joint (the ball-shaped femoral head and the socket in the pelvis) are then removed, and the femoral head is then replaced with a metal stem that goes into the hollow center of the thigh bone. A metal or ceramic ball or sphere is then attached to the top of this stem. The damaged hip socket is then replaced with a metal or plastic cup, called and acetabular cup, which may have a liner made of plastic, ceramic, or metal.

The artificial inserted components are then carefully positioned and secured using specialized surgical cement or techniques that allow for bone growth into the implant surface for stability. Once the implants are in place, the surgeon carefully repositions the muscles and tissues around the new joint and closes the incision with sutures or staples.

While the embodiments disclosed herein are described in use in the environments of a hip arthroplasty or a shoulder suture anchor insertion, it should be noted that the system may be utilized in any type of surgery and is capable of being employed and/or adapted for use in any surgical procedure where it is desirable to insert or place a component in an anatomical space with minimal effort and trauma to the patient. Furthermore, the embodiments disclosed herein may be used in any insertion of a device or component into a space where it may be advantageous to reduce the friction and difficulty of insertion. Thus the various embodiments and aspects disclosed herein are not limited to use in the surgical arts, but rather are applicable to a broad spectrum of human activity without departing from the scope of the embodiment disclosed herein.

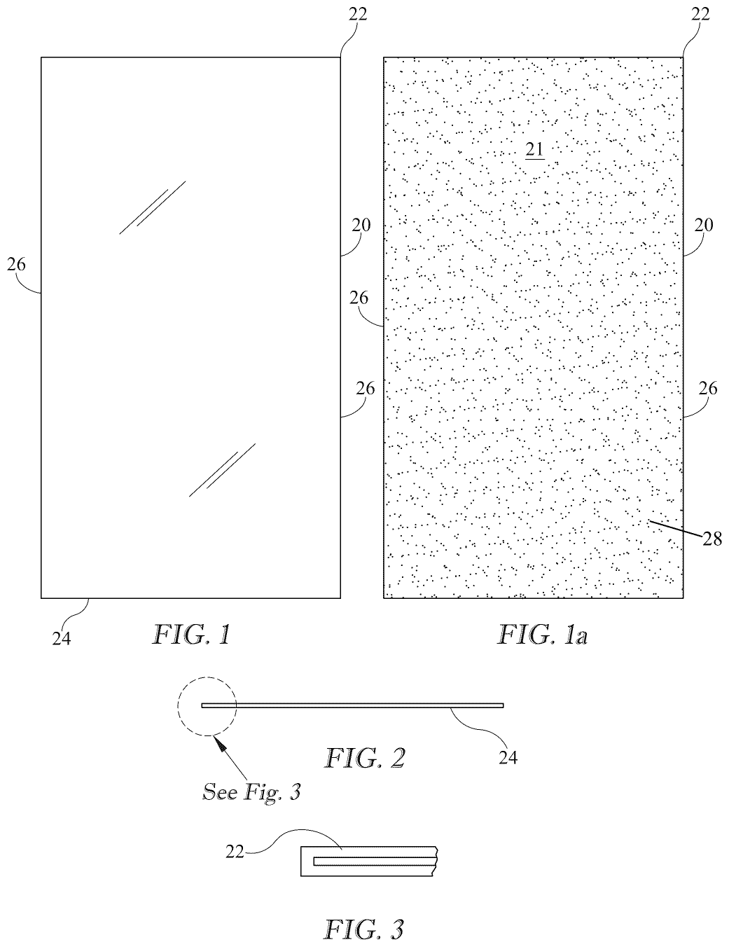

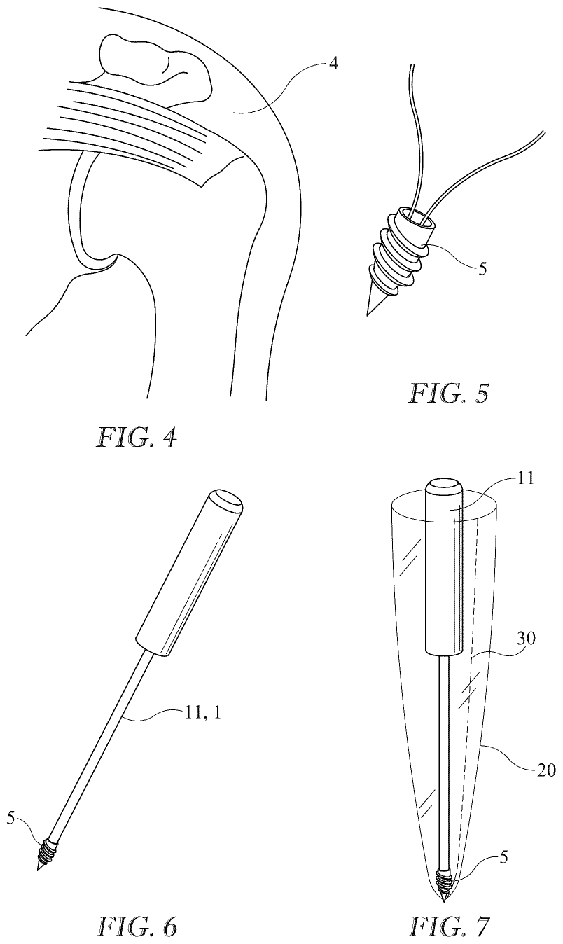

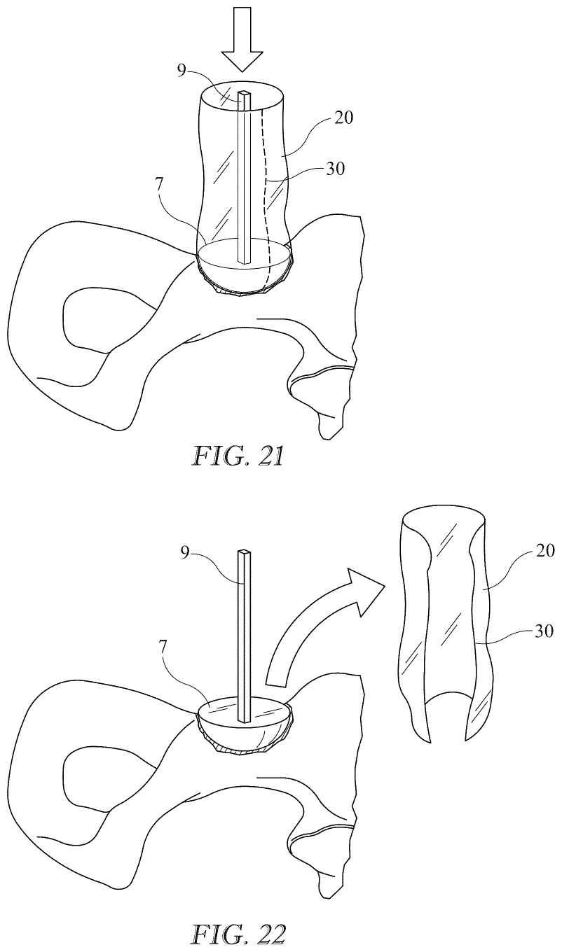

Referring now to the drawing FIGS. 1-20 and in accordance with various aspects and embodiments, herein is described a novel surgical component insertion system 10 for use in inserting a component 1 into an anatomical space 3. The components 1 depicted in FIGS. 4-11 and 17-22 include a suture anchor 5, and an acetabular cup 7, but one of ordinary skill will recognize that a broad array of components 1 may be protected and inserted into an anatomical space 3 when using the system 10 disclosed herein.

System 10 comprises a component pouch 20 having at least one opening or open end 22 for inserting and covering a component 1 during insertion. Pouch 20 may be constructed of a wide variety of low-friction, non-reactive materials, including but not limited to polyethylene films, ultra-high molecular weight (UHMW) polyethylene films, slippery UHMW polyethylene films, electrically conductive carbon-filled UHMW films, polypropylene films, flame retardant polypropylene films, nylon films, wear-resistant nylon films, PTFE (polytetrafluoroethylene) films, slippery PTFE films, PVC films, fluorinated ethylene propylene (FEP) films, ethylene tetrafluoroethylene (ETFE) films, perfluoroalkoxy (PFA) films, polyester films, and high modulus polyethylene films such as Dyneema® composite fabric. In some aspects and embodiments pouches 20 are constructed of films or materials that tear along a single plane, or that tear “evenly” when subjected to sufficient tension. Furthermore, and as best depicted in FIG. 1a, pouch 20 low-friction film material may include a textured or dimpled exterior surface 21 to aid in grasping and manipulating pouch 20 during use.

Pouches 20 may be assembled by modern manufacturing techniques, including ultrasound welding techniques and injection molding techniques. Injection molding techniques are particularly useful where a particular shape is desired for pouch 20 to accommodate a specific component shape. It should be understood that the process used to manufacture pouches 20 is not limiting of the embodiments disclosed and claimed herein. Furthermore, pouches may be manufactured to tear along a predetermined orientation, line, or shape, as necessitated by the physical shape of the component 1 being implanted.

By utilizing pouches 20 manufactured of low-friction, or predetermined friction high-strength film materials such as those disclosed herein above for enclosing implanted components 1, a surgeon may reduce trauma to muscles and tissue, reduce contamination of surgical sites, reduce the aggregation of debris from tissues, and minimize contamination from operating personnel such as surgeons, surgical technicians, physician's assistants and the like. The use of low-friction material for construction pouches 20 also increases the ease of component placement in the anatomical space, since the enclosed implant is easier to position. In some aspects and embodiments pouches may be manufactured from material with a specified frictional coefficient and/or tearing strength based on the specific surgical application desired. In some embodiments, pouches 20 may have sufficient friction to enable a surgeon to move or position the implant 1 and have pouch 20 move with it, thereby requiring a material with a higher coefficient of friction. In other embodiments, the surgeon may require the ability to manipulate or position the implant 1 by pulling pouch 20. In these embodiments pouch 20 may be constructed of a material that has a higher resistance to tear—a higher modulus polypropylene for example, thereby permitting the implant to be moved by manipulation of pouch 20. Additionally, in some embodiments pouches 20 may have a texture that varies along sections of pouches 20 to affect implant 1, pouch interaction. For example, as best seen in FIG. 1a the material used to manufacture pouches 20 may have three-dimensional texture such as raised bumps, ridges, or hemispherical bumps 28 that cause a decrease in surface area contact and therefore a decrease in friction. This feature may be varied along specific sections or zones of pouches 20 such that there is greater friction in predetermined areas of pouches 20 and less friction in other predetermined areas of pouches 20 depending on a desired application. In these embodiments pouches 20 may be constructed having predetermined areas or zones of friction depending on the type of surgery being performed and the specific implant 1 being inserted.

In some applications pouches 20 may be used to deliver and place arthroscopic items, for example a posterior bone block in shoulder 4 arthroscopy procedures. Additionally, pouches 20 may be used for delivery of implants 1 in a wide variety of orthopedic trauma procedures and percutaneous or minimally invasive procedures such as minimally invasive spinal fusion.

A yet further advantage is found in the use of pouches 20 for implantation of components 1 that benefit from preservation in a moist environment, such as allograft tissue. In a yet further application, pouches 20 may be utilized by a surgeon for the removal of tissue while protecting the surrounding anatomy. One exemplary but non-limiting application is the removal of a portion of intestine by grasping it with, and enclosing it in pouch 20, which may reduce the risk of infection caused by intestinal contents contaminating the anatomical space 3.

In some aspects and embodiments as best depicted in FIGS. 1-20, pouch 20 comprises a low-friction film that may be manufactured in a variety of shapes and configurations to accommodate a plurality of instruments and/or components. FIGS. 1-3 depict a generally rectangular pouch 20 having an open end 22, a bottom 24, and two opposed sides 26. In these embodiments for use in an acetabular cup 7 insertion, rectangular pouch 20 may have a film thickness of approximately 0.25 mm, a height of 230 mm, and a width of 127 mm, but a wide variety of pouch 20 thicknesses and dimensions may be used to construct pouch 20 depending upon the size and shape of the component or instrument being inserted.

In some aspects and embodiments pouch 20 may be manufactured to have a shape that accommodates various physical features and contours of component 1 to minimize extraneous material and facilitate a surgeon's ability to view component 1 during the insertion and positioning procedure. Furthermore, pouches 20 may be manufactured to have two open ends 22 where specific surgical applications require it.

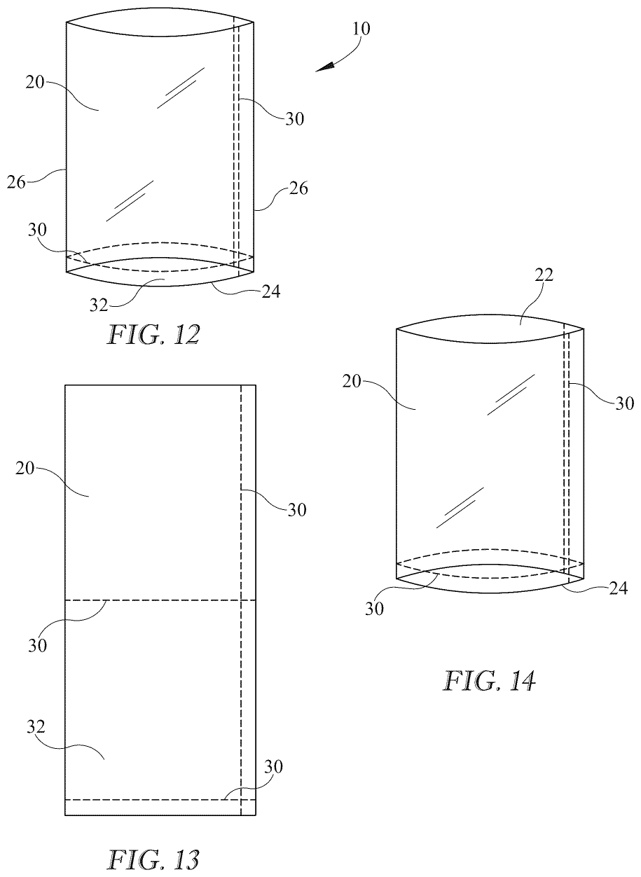

Referring now to those embodiments depicted in FIGS. 12-16e, a surgical component insertion system 10 pouch 20 can include a plurality of perforations 30 or perforated sections 32 arranged in various positions on pouch 20. In some exemplary embodiments as shown in FIGS. 12-14 a generally rectangular pouch 20 includes at least one perforation 30 proximate side 26 along a substantial portion of the length of pouch 20. Furthermore, a perforation 30 may be provided proximate the bottom 24 edge of pouch 20, again along a substantial portion thereof. Perforations 30 may be customized in their spacing (frequency) and size (length) such that a specified or predetermined amount of force must be applied to pouch 20 to separate pouch 20 at the perforations 30. This feature enables the construction of a pouch 20 that is strong enough to retain its integrity during the component 1 insertion portion of a procedure, while being relatively easy to remove once proper insertion and placement are confirmed by the surgical team. In these aspects and embodiments pouch 20 may be readily pulled away from component 1 once it is in position, since the perforations permit pouch to tear and be extracted readily from anatomical space 3.

It should be noted that pouch 20 perforations 30 may be formed into pouch 20 material prior to construction of the pouch 20. Perforations 30 may also be formed after pouch 20 is formed, by use of a perforation wheel or the equivalent. Additionally, pouches 20 may be constructed with perforations 30 formed during the manufacturing process. Perforations 30 may be provided in a plurality of lengths and a range of frequency or spacing to achieve a desired tearing force for separation of pouches 20 depending upon a desired surgical application as required. Furthermore, in accordance with some aspects and embodiments perforations 30 may be oriented in any tearing orientation necessary to facilitate easy removal of pouch 20 once implant 1 is properly positioned.

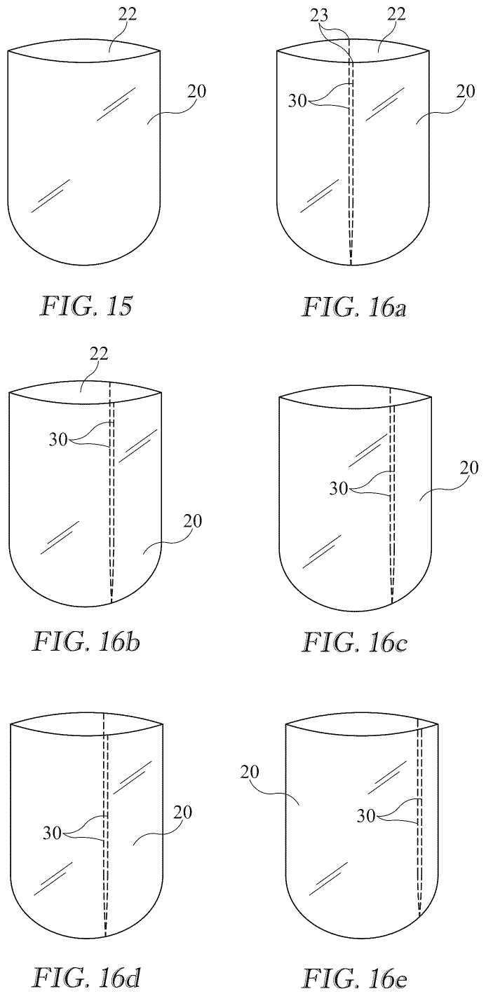

In some embodiments as seen in FIGS. 15-16e pouch 20 may be shaped to form cone, a truncated cone having chamfered or rounded edges, or a frusto-conical pouch 20. In these exemplary embodiments perforations 30 may be disposed at various sections or areas of pouch 20. For example, FIG. 16a depicts a perforation 30 that runs from an edge 23 of open end 22 of pouch 20 to the “bottom” or vertex of semi-conical pouch 20 and back up to edge 23. This embodiment allows pouch 20 to tear in half, or in two relatively identical sections, to facilitate removal of pouch 20 once component 1 is positioned. Similarly, FIGS. 16 b-e depict a plurality of alternative perforation 30 arrangements, wherein pouch 20 may be separated into two unequal portions along perforations 30 depending upon particular surgical requirements for a given component 1 and anatomical space 3.

In other embodiments, FIG. 16b depicts pouch 20 having perforation 30 located such that pouch 20 may be torn into two segments, one being approximately ⅔ of the entire pouch 20 and the other being approximately ⅓ thereof. Similarly, FIG. 16c provides a perforation 30 located so that pouch 20 to be torn into two segments, one being approximately ¾ of the entire pouch 20, and the other being approximately ¼ thereof. FIGS. 16d and 16e depict several other embodiments having perforations 30 dividing pouch 20 into several segments of varying sizes that may be utilized for different component 1 sizes and orientations. As seen from these embodiments a plurality of perforation 30 orientations may be used to provide a surgeon the ability to tear pouches 20 into sections of varying sizes, depending upon the particular surgical requirements. In one exemplary embodiment, perforations 30 may be provided in pouch 20 so that pouch 20 tears into three or more separate pieces when sufficient stress is applied. Furthermore, in some aspects and embodiments perforations 30 may be curvilinear or shaped in any desired fashion along the surface of pouches 20 to provide separation of pouch 20 material along a specific area or areas that mimics or mirrors a contour or shape of component 1.

Referring now to the embodiments and aspects depicted in FIGS. 4-11, pouch 20 is depicted in use in implantation of a suture anchor 5 in a shoulder 4 surgery. In these embodiments pouch 20 may advantageously be generally conical in shape and may have a hole or aperture 32 provided at a point near the vertex of conical pouch 20 through which a sharp end of suture anchor 5 may protrude. As best shown in FIGS. 5, 6 and 7, suture anchor 5 includes a plurality of helical threads that may cause trauma to tissue during insertion. Once a suture is secured to anchor 5, an insertion tool 11 is used to grasp anchor 5 for insertion, and pouch 20 is placed over anchor 5 and tool 11 with the end of anchor 5 protruding through aperture 32 of pouch 20.

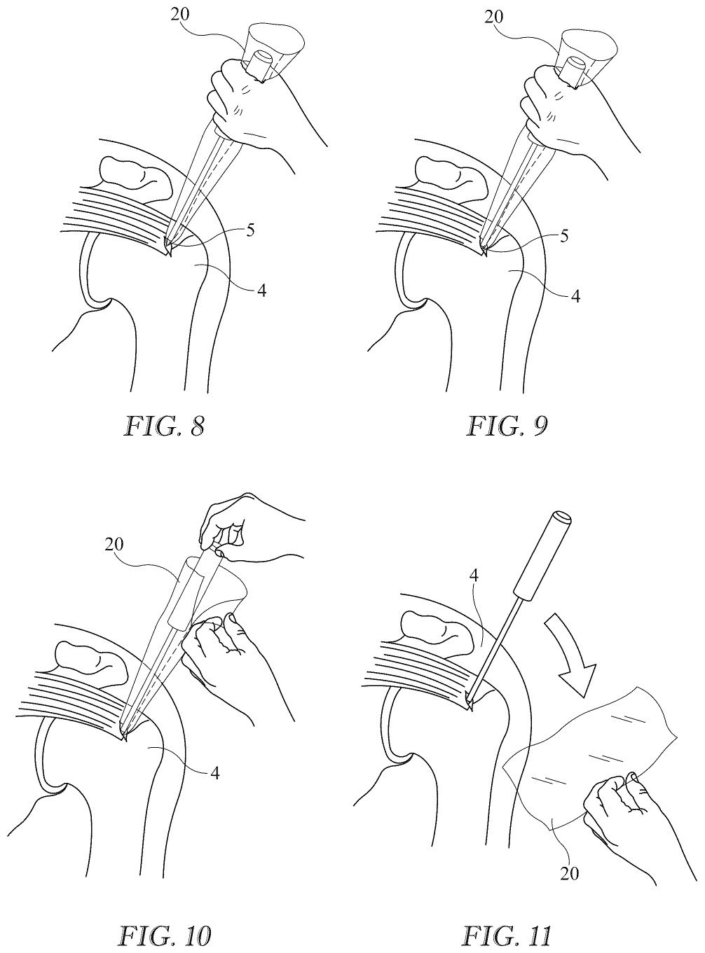

FIGS. 8-11 depict anchor 5 being inserted and positioned in shoulder 4, with pouch 20 surrounding anchor 5 and tool 11 to protect shoulder 4 as anchor 5 is placed. Once anchor 5 is secured, pouch 20 may be torn away by separating pouch 20 into pieces along perforations 30, as shown in FIGS. 10 and 11.

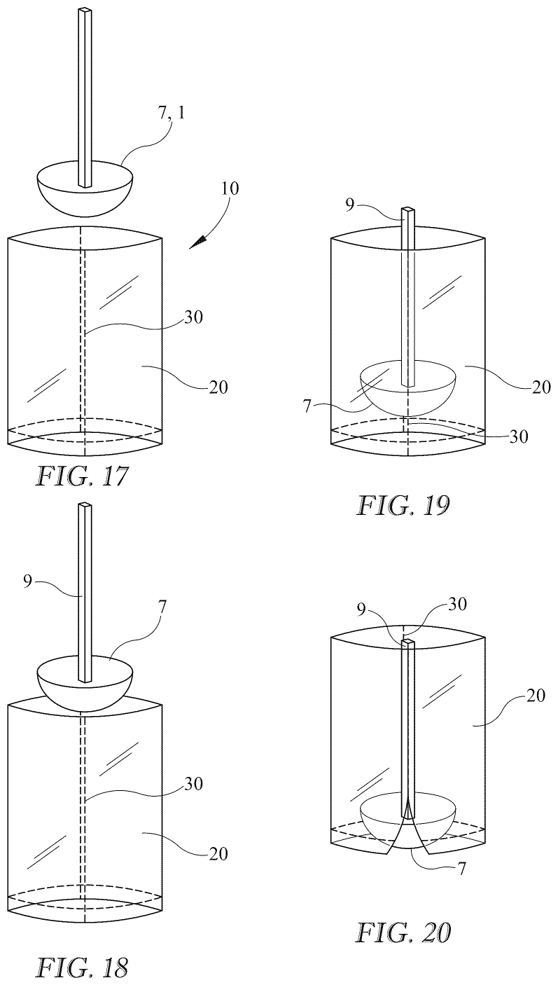

In some aspects and embodiments as best seen in FIGS. 17-22, system 10 is employed with an insertion tool 9 for inserting and placing an acetabular cap 7. Cap 7 is secured to insertion tool 9 and placed in pouch 20. Once the insertion site for cap 7 is prepared, pouch 20 is inserted in anatomical space 3 and acetabular cap 7 is then positioned in the insertion site. Pouch 20 may then be torn along perforations 30 and removed from anatomical space 3.

In some additional aspects and applications, pouch 20 may be utilized as a protective covering for a component 1 during its manufacture and packing process. For example, an implant manufacturer may package their sterile implant already inserted in sterile pouch 20, thereby enabling a surgical team to simply open the sterile package and insert the component 1. This feature of system 10 provides a significant advantage over other systems since there is no extraneous handling of component 1 prior to its use, thereby reducing the risk of contamination and infection during handling.

While several embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It is to be understood that the embodiments are not limited in its application to the details of construction and the arrangement of components set forth in the description or illustrated in the drawings. The invention is capable of other embodiments and of being practiced or of being carried out in various ways. Unless limited otherwise, the terms “connected,” “coupled,” “in communication with,” and “mounted,” and variations thereof herein are used broadly and encompass direct and indirect connections, couplings, and mountings. In addition, the terms “connected” and “coupled” and variations thereof are not restricted to physical or mechanical connections or couplings.

While the present invention has been shown and described herein in what are considered to be the preferred embodiments thereof, illustrating the results and advantages over the prior art obtained through the present invention, the invention is not limited to those specific embodiments. Thus, the forms of the invention shown and described herein are to be taken as illustrative only and other embodiments may be selected without departing from the scope of the present invention, as set forth in the claims appended hereto.

We claim:

1. An insertion system for use by a surgeon for placing a surgical component into an anatomical space comprising:
a pouch having an open end for insertion of said surgical component, said pouch having at least one perforation along a portion thereof, said perforation enabling said pouch to tear open for removal once said component is inserted in said anatomical space.

2. The system of claim 1 comprising:
a plurality of shaped perforations disposed in said pouch, whereby said pouch is readily torn and removed when pulled by said surgeon.

3. The system of claim 1 wherein said pouch is generally a four sided rectangle having opposed long sides and one open end and one closed short side, and wherein said open end is located along one of said short sides.

4. The system of claim 3 comprising:
a plurality of perforations along a one of said long sides.

5. The system of claim 3 comprising:
a plurality of perforations along a one of said long sides and said closed short side.

6. The system of claim 1 comprising:
said pouch having a generally cylindrical shape having an open end and a closed end.

7. The system of claim 6 wherein said perforations extend around a portion of said pouch from two points on said open end and around said closed end.

8. The system of claim 1 wherein said pouch is comprised of a UHMW polyethylene sheet.

9. The system of claim 8 wherein said polyethylene sheet is approximately 0.25 mm in thickness.

10. The system of claim 1 wherein said pouch is comprised of a polytetrafluoroethylene sheet.

11. The system of claim 10 wherein said polytetrafluoroethylene sheet is approximately 0.25 mm in thickness.

12. The system of claim 10 wherein said pouch has an exterior that includes a textured surface portion for enhancing grip on the exterior thereof.

13. The system of claim 1 wherein said pouch is comprised of a nylon film sheet.

14. The system of claim 1 wherein said pouch is comprised of a fluorinated ethylene propylene film sheet.

15. The system of claim 1 wherein said pouch is comprised of a polyvinyl chloride film.

16. The system of claim 1 wherein said pouch is comprised of material having a linear tear pattern.

17. The system of claim 1 comprising:
an aperture in a portion thereof through which a portion of said component may be inserted for placement into said anatomical space.

18. The system of claim 1 wherein said pouch is generally frusto-conical in shape.

19. An insertion system for use by a surgeon for placing a surgical component into an anatomical space comprising:
a sacrificial container having an open end for insertion of said surgical component into said container, said pouch having a plurality of perforations along segments thereof, said perforations enabling said pouch to tear open for removal once said component is inserted in said anatomical space.

20. The system of claim 19 wherein said container comprises a textured exterior surface.

21. The system of claim 20 wherein said textured surface includes a plurality of raised elements, said raised elements being variable along predetermined sections of said container, thereby varying friction between said container and said anatomical space.

22. The system of claim 19 wherein said sacrificial container is comprised of a polymer film.

23. The system of claim 19 wherein said sacrificial container is shaped to accommodate said surgical component.
